(19) United States
(12) Patent Application Publication
(10) Pub. No.: US 2026/0114708 A1
(43) Pub. Date: Apr. 30, 2026

(54) SYSTEM AND METHOD FOR GENERATING ENDOSCOPIC IMAGE

(71) Applicant: Point Robotics Medtech Inc., Hsinchu County (TW)

(72) Inventors: Jiun-Yu Weng, Hsinchu County (TW); SHIH-YUAN CHEN, Hsinchu County (TW); HSIN CHAN, Hsinchu County (TW)

(21) Appl. No.: 19/059,270

(22) Filed: Feb. 21, 2025

(30) Foreign Application Priority Data
Oct. 30, 2024 (TW) 113141382

(51) Int. Cl. A61B 1/00; A61B 1/05; A61B 34/10; A61B 34/20; A61B 34/30; A61B 90/00

(52) U.S. Cl. CPC A61B 1/00009; A61B 1/00105; A61B 1/00181; A61B 1/05; A61B 34/10; A61B 34/20; A61B 34/30; A61B 90/08; A61B 2034/301; A61B 2090/0807

(57) ABSTRACT

A system and a method for generating an endoscopic image are provided. The system includes an endoscope assembly, a robot device, and a processing device. The endoscope assembly is suitable for being used with a surgical instrument and includes a plurality of image capturing elements. The image capturing elements capture a plurality of current images from different viewing angles. One of the current images includes a first image part and a second image part. The first image part is an image of a part of the surgical instrument, and the second image part is an image of a part of the surgical site that is not obscured by the part of the surgical instrument. At least one of the current images further includes a third image part. The processing device is configured to replace the first image part with the third image part, to obtain a processed image.

### CROSS-REFERENCE TO RELATED PATENT APPLICATION

This application claims the benefit of priority to Taiwan Patent Application No. 113141382, filed on Oct. 30, 2024. The entire content of the above identified application is incorporated herein by reference.

### FIELD OF THE DISCLOSURE

The present disclosure relates to a system and a method for generating an image, and more particularly to a system and a method for generating an endoscopic image.

### BACKGROUND OF THE DISCLOSURE

Spinal decompression surgery is commonly used to remove spine spurs which compress nerves or herniated intervertebral discs which that squeeze nerves, thereby freeing up space in the nerve cavity to relieve pressure on the nerve roots and alleviate the patient's pain. Generally, a doctor can perform spinal decompression surgery by using a microscope. The doctor first inserts a cannula into the patient's surgical site, observes enlarged images of the surgical area through the microscope, and then performs the surgery by viewing real-time images. However, the volume of the microscope inevitably occupies a certain space of the operating room, thus limiting the doctor's activity range. Furthermore, the view field of the surgical site seen through the microscope may be blocked by surgical instruments, the doctor is unable to see the entire surgical site, thereby affecting the stability and accuracy of the surgical process.

Therefore, how to overcome the above-mentioned problem through an improvement in structural design has become an important issue to be addressed in the related art.

### SUMMARY OF THE DISCLOSURE

In response to the above-referenced technical inadequacy, the present disclosure provides a system and a method for generating an endoscopic image.

In order to solve the above-mentioned problems, the present disclosure provides a system for generating an endoscopic image which includes an endoscope assembly, a robot device, and a processing device. The endoscope assembly is suitable for being used with a surgical instrument. The endoscope assembly includes a plurality of image capturing elements. The robot device is connected to the endoscope assembly. The robot device is configured to move the endoscope assembly to vicinity of a surgical site, such that the plurality of image capturing elements capture a plurality of current images from different viewing angles, respectively. One of the current images includes a first image part and a second image part. The first image part is an image of a part of the surgical instrument. The second image part is an image of a part of the surgical site that is not obscured by the part of the surgical instrument. At least one of the current images further includes a third image part. The third image part and the first image part correspond to a same area or a same position of the surgical site. The processing device is electrically connected to the endoscope assembly. The processing device is configured to: identify positions and contours of the surgical instrument in the plurality of current images; divide the plurality of current images to separate the first image parts, the second image parts, and the third image parts of the plurality of current images; and replace one of the first image parts with one of the third image parts, so as to obtain a processed image.

In order to solve the above-mentioned problems, the present disclosure provides a method of generating an endoscopic image, which includes the following steps. Providing a robot device to connect an endoscope assembly and move the endoscope assembly to vicinity of a surgical site, in which the endoscope assembly is suitable for being used with a surgical instrument. Providing a plurality of image capturing elements that are disposed on the endoscope assembly to respectively capture a plurality of current images of the surgical site from different viewing angles, wherein one of the current images includes a first image part and a second image part, the first image part is an image of a part of the surgical instrument, the second image part is an image of a part of the surgical site that is not obscured by the part of the surgical instrument, at least one of the current images further includes a third image part, and the third image part and the first image part correspond to a same area or a same position of the surgical site. Providing a processing device that is electrically connected to the endoscope assembly to: identify positions and contours of the surgical instrument in the plurality of current images, divide the plurality of current images to separate the first image parts, the second image parts, and the third image parts of the plurality of current images, and replace one of the first image parts with one of the third image parts, so as to obtain a processed image.

Therefore, with the disclosed system and the disclosed method for generating an endoscopic image, through dividing, identifying, and combining image parts of the images of the surgical site, the image part in which the surgical site is blocked by the surgical instrument can be replaced with another image part in which the surgical site is not blocked by the surgical instrument, and the doctor can see the whole surgical site. Therefore, the system for generating the endoscopic image can be used without a microscope, so that space in the operating room can be reserved to benefit surgical operations.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a system for generating an endoscopic image according to the present disclosure;

FIG. 2 is a schematic view of an endoscope assembly according to the present disclosure;

FIG. 3 is schematic exploded view of the endoscope assembly according to the present disclosure;

FIG. 4 is a schematic bottom view of an inner sleeve of the endoscope assembly according to the present disclosure;

FIG. 5 is a schematic view of an image capturing element according to the present disclosure;

FIG. 6 is a schematic top view of a current image of a surgical site generated by the system for generating the endoscopic image according to the present disclosure;

FIG. 7 is a schematic top view of a first image part of the surgical site generated by the system for generating the endoscopic image according to the present disclosure;

FIG. 8 is a schematic top view of another current image of the surgical site generated by the system for generating the endoscopic image according to the present disclosure;

FIG. 9 is a schematic top view of a processed image of the surgical site generated by the system for generating the endoscopic image according to the present disclosure;

FIG. 10 is a flowchart of steps S1 to S5 of a method of generating an endoscopic image according to the present disclosure;

FIG. 11 is a flowchart of steps S51 to S54 of the method of generating the endoscopic image according to the present disclosure; and

FIG. 12 is a flowchart of steps S11 to S16 of the method of generating the endoscopic image according to the present disclosure.

### DETAILED DESCRIPTION OF THE EXEMPLARY EMBODIMENTS

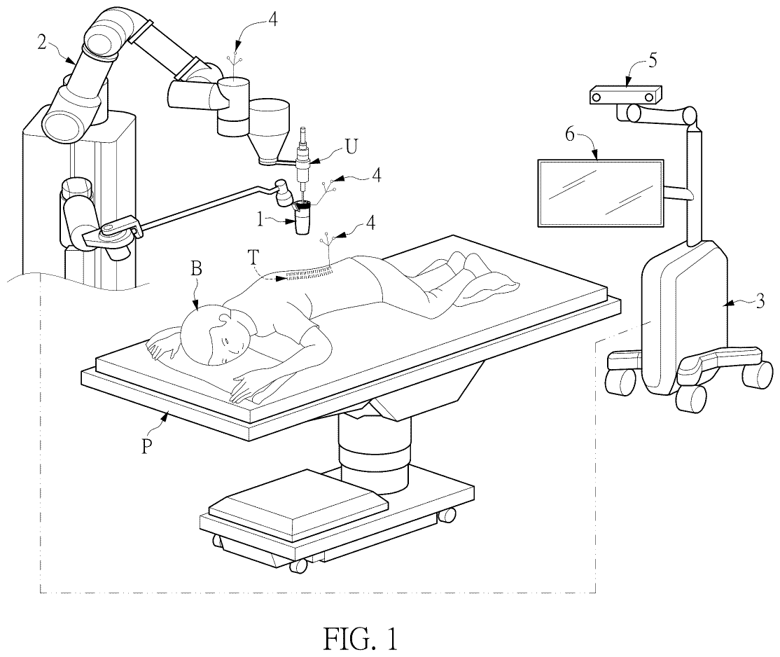

Referring to FIG. 1, an embodiment of the present disclosure provides a system for generating an endoscopic image which includes an endoscope assembly 1, a robot device 2, and a processing device 3. The robot device 2 includes a plurality of mechanical arms capable of moving with multiple degrees of freedom. The robot device 2 is connected to the endoscope assembly 1 and a surgical instrument U. The endoscope assembly 1 can be used with the surgical instrument U for performing the surgery on a patient B lying prone on an operating table P. The processing device 3 is electrically connected to the endoscope assembly 1. The processing device 3 can include, for example, a processor and a memory. However, the present disclosure is not limited thereto. The processor can be, for example but not limited to, a programmable logic controller, a microprocessor, an integrated microcontroller circuit, a central processing unit, etc. The memory can be, for example but not limited to, a random access memory (RAM), a read only memory (ROM), a flash memory, a hard disk, or other storage devices that can be used to store data.

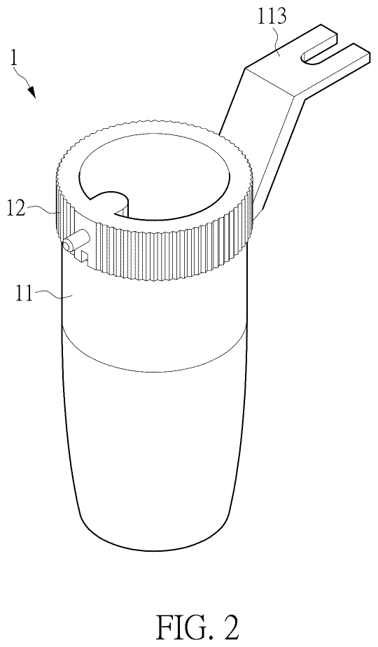

Reference is made to FIG. 2 to FIG. 4. The endoscope assembly 1 further includes an outer sleeve 11 and an inner sleeve 12 that are detachable from each other. The outer sleeve 11 and the inner sleeve 12 can be assembled and disassembled with each other. The outer sleeve 11 has a first opening 111 and a second opening 112 that communicate with each other. The inner sleeve 12 is disposed at the first opening 111 and is located inside the outer sleeve 11. Each of the outer sleeve 11 and the inner sleeve 12 has a hollow cylindrical shape. An outer wall 121 of the inner sleeve 12 has a plurality of grooves 12V, and the plurality of grooves 12V are arranged in a ring along the outer wall 121. Furthermore, the outer sleeve 11 further includes a connection portion 113. The robot device 2 can grip the connection portion 113 through the mechanical arms to move the outer sleeve 11. When the inner sleeve 12 is assembled onto the outer sleeve 11, the plurality of grooves 12V are distributed around the inner sleeve 12. A limiting block 11B on an inner wall of the outer sleeve 11 is engaged into one of the grooves 12V and limits the inner sleeve 12 to a specific azimuth, preventing the inner sleeve 12 from being rotated after being installed on the outer sleeve 11. If a user wants to change the azimuth of the inner sleeve 12, the inner sleeve 12 needs to be disassembled from the outer sleeve 11 first and then reinstalled onto the outer sleeve 11 in a different azimuth, allowing the limiting block 11B to be engaged in another groove 12V.

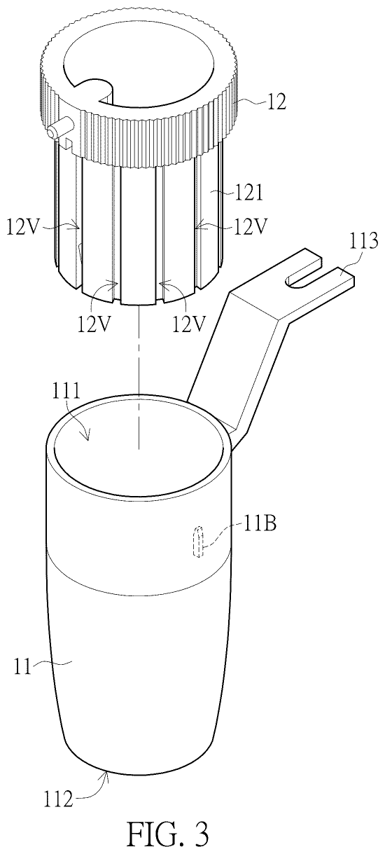

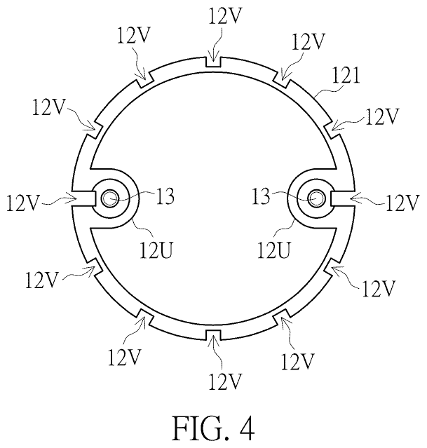

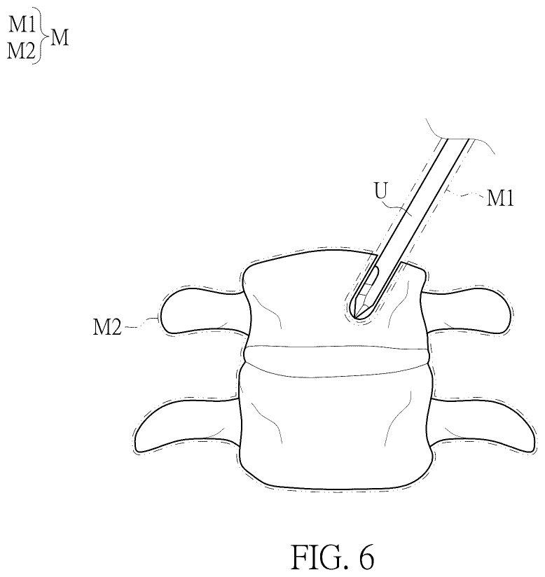

Reference is made to FIG. 3 and FIG. 4. The endoscope assembly 1 includes a plurality of image capturing elements 13. A quantity of the image capturing elements 13 is not limited in the present disclosure. In the embodiment of the present disclosure, the quantity of the image capturing elements 13 is two. The two image capturing elements 13 are disposed in two receiving grooves 12U of the inner sleeve 12, respectively. The two receiving grooves 12U are located along a diameter direction of the inner sleeve 12. A distance between the two image capturing elements 13 is approximately less than a diameter of the inner sleeve 12. That is, the two image capturing elements 13 are disposed at opposite ends on a same diameter of the inner sleeve 12, with the image capturing elements 13 parallelly disposed. In addition, when the inner sleeve 12 and the outer sleeve 11 are assembled with each other, the inner sleeve 12 and the outer sleeve 11 are coaxially arranged and communicate with each other. The two image capturing elements 13 are located in the outer sleeve 11 and face the second opening 112 of the outer sleeve 11. Through the design that one of the two image capturing elements 13 is symmetric to the other, captured images of a surgical site T can be used for displaying the maximum area of the surgical site T. It should be noted that the surgical site T refers to a spinal site of the patient B. FIG. 6 to FIG. 9 show schematic top views of the spinal site.

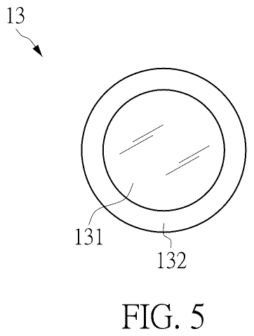

Reference is made to FIG. 5. Each of the image capturing elements 13 includes a lens 131 and a light emitting element 132. The light emitting element 13 can be a light-emitting diode (LED) element and is disposed around the lens 131. The lens 131 can be an endoscope lens or a camera lens, but the present disclosure is not limited thereto. While the image capture elements 13 are used, the light emitting elements 132 serve as light sources to illuminate the place to which the lenses 131 are heading, so that a user (i.e., the doctor) can clearly see specific features of the surgical site T through the lenses 131.

Reference is further made to FIG. 1. The system for generating the endoscopic image further includes a plurality of navigation marker frames 4 and a tracker 5. The plurality of navigation marker frames 4 are disposed on the robot device 2 (i.e., the robotic arm holding the surgical instrument U), the endoscope assembly 1, and the surgical site T of the patient B. Each of the navigation marker frames 4 includes a dynamic reference frame (“DRF”) and a plurality of marker elements arranged on the dynamic reference frame. For example, the marker element is an optical element, such as a reflective ball, and the tracker 5 can be, for example, an optical tracker. The tracker 5 is electrically connected to the processing device 3. The navigation marker frames 4 can be used as spatial positioning points for establishing a spatial coordinate system. The tracker 5 can sense, detect and record coordinate positions of the optical elements on the navigation marker frames 4, and transmit the information (i.e., the coordinate positions of the optical elements) to the processing device 3 for calculation and/or storage.

Each of the navigation marker frames 4 is a deformable device. Specifically, the shape of the DRF of each navigation marker frame 4 can be adjusted. Therefore, the user can freely adjust the shape of the DRF according to the position at which the navigation marker frame 4 is located, so as to achieve the purpose of identifying different instruments. In addition, the marker elements of the navigation marker frame 4 and the tracker 5 are illustrated by taking them as optical sensing components, but the present disclosure is not limited thereto. In other embodiments, the marker elements of the navigation marker frame 4 and the tracker 5 can be used for establishing the spatial coordinate system through electromagnetic sensing.

The robot device 2 is configured to guide the surgical instrument U and the endoscope assembly 1 to move to the surgical site T based on the spatial coordinate system, and then the surgical instrument U is put down through the inner sleeve 12 and the outer sleeve 11 to enter into the surgical site T for surgical operation. The surgical instrument U is enclosed within the inner sleeve 12 and the outer sleeve 11. The processing device 3 obtains position information of the surgical instrument U in the surgical site T and images corresponding to the position information through the lenses 131 of the endoscope assembly 1.

The system for generating the endoscopic image further includes a display device 6 that is electrically connected to the processing device 3. For example, the display device 6 can include a screen and a buzzer (not shown in the figures). The processing device 3 builds a three-dimensional anatomy model near the surgical site T by integrating pre-obtained medical images, such as imagines from computed tomography (CT) or magnetic resonance imaging (MRI). The three-dimensional anatomy model can be displayed on the screen of the display device 6. The processing device 3 acquires images near the surgical site T through the image capturing elements 13 of the tube scope assembly 1, and the images and the three-dimensional anatomy model are together displayed on the screen of the display device 6. Therefore, the doctor can simultaneously obtain images about information of the whole surgical operation space, position information of the surgical instrument U relative to the surgical site T, and real-time photographing of the surgical site T. Then, under guidance of a surgical navigation system, the doctor may perform the surgery in accompany with the robot device 2.

When the processing device 3 detects that the surgical instrument U deviates from a predetermined surgical path, the processing device 3 provides a warning signal in the form of an image or a sound (e.g., a red flashing on the screen or a specific sound from a buzzer) to medical personnel through the display device 6 for immediately alerting medical staff, thereby increasing positioning reliability and navigation accuracy.

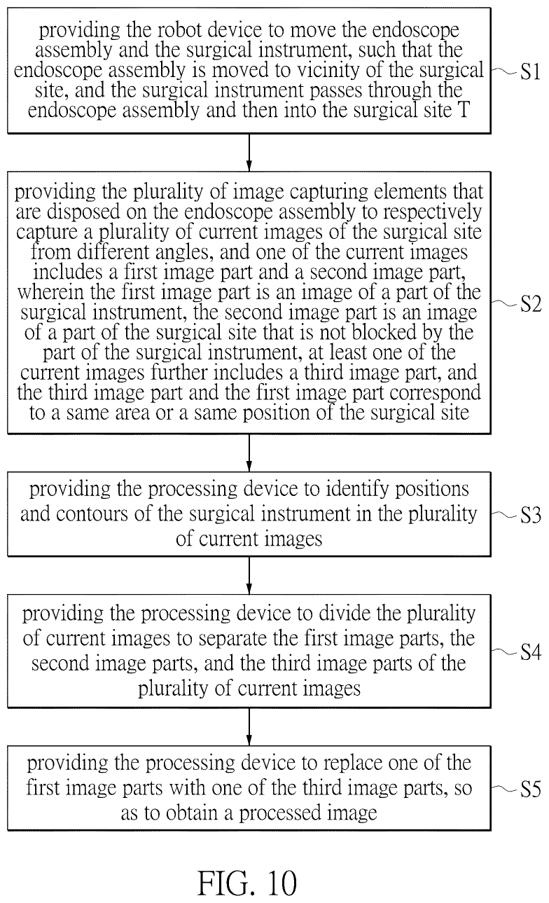

In order to further illustrate the specific operational method of the system for generating the endoscopic image in the present disclosure, reference is made to FIG. 10. The present disclosure provides a method of generating the endoscopic image, which can be implemented through the aforementioned system for generating the endoscopic image. As shown in FIG. 10, the method of generating the endoscopic image includes steps S1 to S5.

Step S1: providing the robot device to move the endoscope assembly and the surgical instrument, such that the endoscope assembly is moved to vicinity of the surgical site, and the surgical instrument passes through the endoscope assembly and then into the surgical site T.

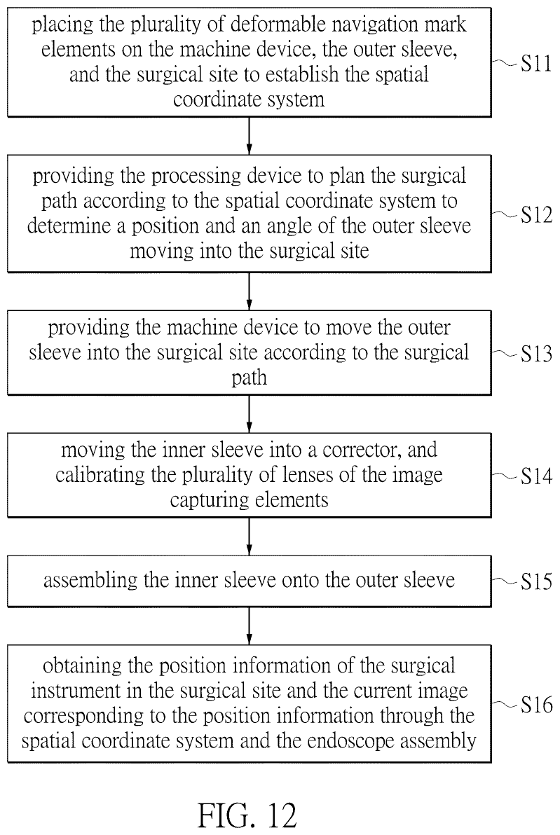

Furthermore, reference is made to FIG. 12, in step S1, the step of moving the endoscope assembly to the surgical site further include steps S11 to S16:

Step S11: placing the plurality of deformable navigation marker frames on the robot device, the outer sleeve, and the surgical site to establish the spatial coordinate system.

Step S12: providing the processing device to plan the surgical path according to the spatial coordinate system to determine a position and an angle at which the outer sleeve enters into the surgical site.

Step S13: providing the robot device to move the outer sleeve to vicinity of the surgical site according to the surgical path.

Step S14: disposing the inner sleeve in a calibrator, and calibrating the plurality of lenses of the image capturing elements.

Step S15: assembling the inner sleeve onto the outer sleeve.

Step S16: obtaining the position information of the surgical instrument in the surgical site and the current image corresponding to the position information through the spatial coordinate system and the endoscope assembly.

Reference is further made to FIG. 10. Step S2: providing the plurality of image capturing elements that are disposed on the endoscope assembly to respectively capture a plurality of current images of the surgical site from different angles, and one of the current images includes a first image part and a second image part. The first image part is an image of a part of the surgical instrument, the second image part is an image of a part of the surgical site that is not blocked by the part of the surgical instrument, at least one of the current images further includes a third image part, and the third image part and the first image part correspond to a same area or a same position of the surgical site.

Step S3: providing the processing device to identify positions and contours of the surgical instrument in the plurality of current images.

Step S4: providing the processing device to divide the plurality of current images to separate the first image parts, the second image parts, and the third image parts of the plurality of current images.

Step S5: providing the processing device to replace one of the first image parts with one of the third image parts, so as to obtain a processed image.

The aforementioned steps will be further explained in detail below. Referring to FIG. 3, the spatial coordinate system is first established based on the navigation marker frames 4 disposed on the endoscope assembly 1, the mechanical arm holding surgical instrument U, and near the surgical site T. The processing device 3 is capable of planning a surgical path based on the spatial coordinate system. By means of the surgical path, the position and the angle at which the outer sleeve 11 of the endoscope assembly 1 as well as the surgical instrument U enters the surgical site T can be determined. After determining the position and the angle of the outer sleeve 11, the robot device 2 is operated to move the outer sleeve 11 into the surgical site T. Then, the inner sleeve 12 is placed into a calibrator (not shown in the figures), and the two lenses 131 of the two image capturing elements 13 disposed on the inner sleeve 12 are calibrated. The purpose of the calibration is to set the focuses of the two lenses 131 at the same plane and to rotate one lens image to have the identical orientation of the other lens image.

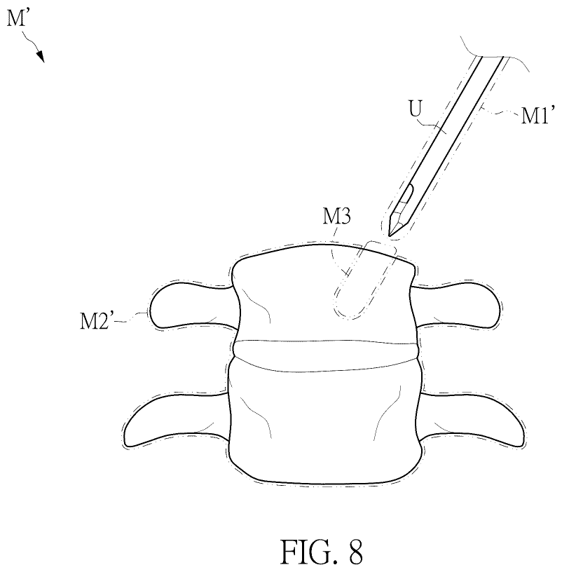

Then, the calibrated inner sleeve 12 is assembled onto the outer sleeve 11, and the robot device 2 is operated to move the surgical instrument U to pass through the endoscope assembly 1 and enter the surgical site T. Through the spatial coordinate system and the endoscope assembly 1, both the position information of the surgical instrument U within the surgical site T and the current image corresponding to the position information are obtained. Specifically, as shown in FIGS. 6 and 8, the two image capturing elements 13 respectively capture two current images M and M′ of the surgical site T through their own lenses 131. Since the positions of the two image capturing elements 13 are different (i.e., the two image capturing elements 13 are parallelly disposed), the two current images M and M′ are captured from different viewing angles. It should also be noted that FIGS. 6 and 8 are merely examples for illustrating operation of the system and the method for generating the endoscopic image provided by the present disclosure and do not represent the actual conditions inside the surgical site T. For instance, the position of the surgical instrument U is not necessarily the same as shown in FIGS. 6 and 8.

Reference is further made to FIGS. 6 and 8. The processing device 3 performs image recognition on the surgical instrument's position and contour within two current images M and M′, so as to segment the two current images M and M′ into multiple parts. For example, through recognition processing by the processing device 3, the current image M in FIG. 6 includes a first image part M1 and a second image part M2, while the current image M′ in FIG. 8 includes a first image part M1′ and a second image part M2′. Each of the first image parts M1 and M1′ represents an image of a part of the surgical instrument U, while the second image parts M2 and M2′ represent areas of the surgical site T that are not blocked by the surgical instrument U. Additionally, the current image M in FIG. 8 further includes a third image part M3, the third image part M3 in FIG. 8 and the first image part M1 in FIG. 6 both correspond to the same area or the same position of the surgical site T.

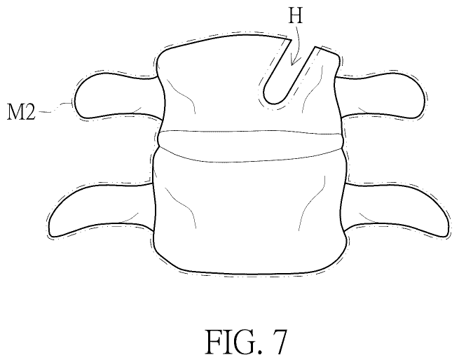

The processing device 3 performs image separation on the two current images M and M′. The first image part M1 of the current image M in FIG. 6 is removed, leaving the second image part M2, as shown in FIG. 7. In FIG. 7, the second image part M2 is retained, while the position that was originally occupied by the first image part M1 in FIG. 6 now is a vacant area H. Then, the third image part M3 is divided from FIG. 8. Since the third image part M3 and the first image part M1 correspond to the same area of the surgical site T, the third image part M3 and the vacant area H in FIG. 7 also correspond to the same area of the surgical site T.

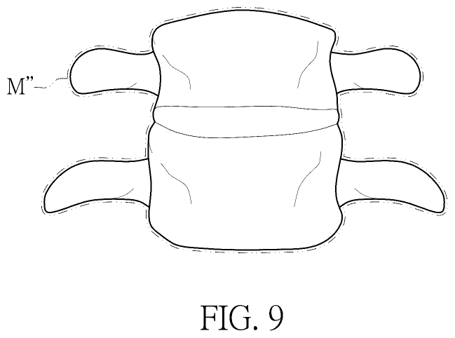

Subsequently, the processing device 3 splices the third image part M3 in FIG. 8 to the vacant area H in FIG. 7 and merge the third image part M3 with the second image part M2, thereby obtaining a processed image M″. That means, as shown in FIGS. 6, 8, and 9, through the images captured from different viewing angles by the two image capturing elements 13, the processing device 3 replaces the first image part M1 in FIG. 6 with the third image part M3 from FIG. 8 to obtain the processed image M″ in FIG. 9. The processed image M″ is an unobstructed field image which is built by combining multiple surgical images from different angles. Therefore, through the system and method for generating the endoscopic image provided by the present disclosure, the image of the surgical instrument U is removed, and the image that is blocked by the surgical instrument U is revealed. To minimize parallax, the processing device 3 performs image processing on the third image part M3 before stitching it to the second image part M2. This processing may involve translating the third image part M3 to capture necessary image parts. Before merging the third image part M3 with the second image part M2, the processing device 3 performs brightness adjustments on both the third image part M3 and the second image part M2 to ensure that no particularly abrupt areas appear in the merged image.

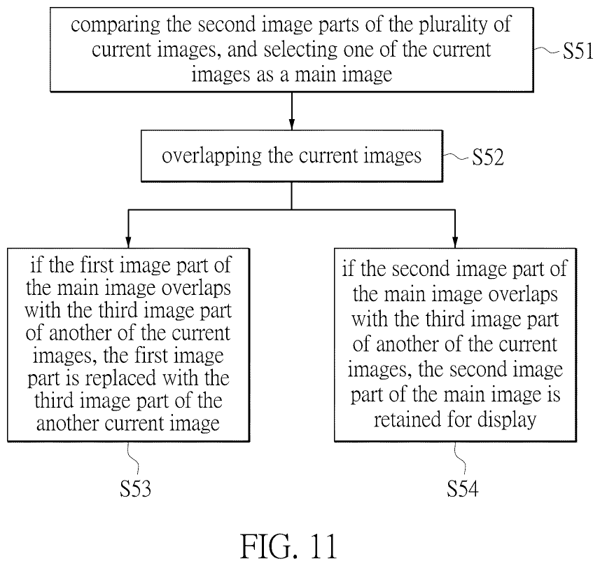

Reference is made to FIG. 11. The step S5 of replacing one of the first image parts with one of the third image parts to obtain the processed image further includes step S51 to step S54:

Step S51: comparing the second image parts of the plurality of current images, and selecting one of the current images as a main image.

Step S52: overlapping the current images.

Step S53: if the first image part of the main image overlaps with the third image part of another of the current images, the first image part is replaced with the third image part of the another current image.

Step S54: if the second image part of the main image overlaps with the third image part of another of the current images, the second image part of the main image is retained for display.

Specifically, as shown in FIGS. 6, 7, and 8, the processing device 3 selects one of the current images to serve as the main screen. For example, the current image M in FIG. 6 serves as the main screen. Then, the processing device 3 cuts out the image portion covering the instrument's position in the main screen (i.e., the first image part M1), creating the vacant area H. In the meantime, the main screen shown in FIG. 7 retains the second image part M2.

The processing device 3 further overlaps the main screen shown in FIG. 7 with another screen shown in FIG. 8 and performs a comparison. When overlapping the two screens, if the third image part M3 of the current image M′ in FIG. 8 overlaps with the vacant area H of the second image part M2 in the main screen in FIG. 7, the processing device 3 fills the vacant area H with the third image part M3. That is, the first image part M1 in FIG. 6 is replaced with the third image part M3 from FIG. 8. On the contrary, if the third image part M3 of the current image M′ in FIG. 8 overlaps with the second image part M2 in FIG. 7 rather than the first image part M1, the processing device 3 does not replace the second image part M2 with the third image part M3 but keeps the second image part M2 on the main screen in FIG. 6 for display.

In other words, the processing device 3 compares the image parts by overlapping the two current images M and M′ to confirm that the third image part M3 in FIG. 8 overlaps with the area blocked by the surgical instrument U in FIG. 6, and then the processing device 3 replaces the image of the surgical instrument U with the third image part M3.

It should be noted that the aforementioned description for steps S51 to S54 is merely an exemplary embodiment. The method of selecting a main screen for image overlapping and comparison is not limited in the present disclosure. In other embodiments, the processing device 3 can also set different weights, such as assigning different weights to the current image M in FIG. 6 and the current image M′ in FIG. 8. For example, the current image M in FIG. 6, which includes the second image part M2 in FIG. 7, can be assigned a high weight, while the current image M′ in FIG. 8 can be assigned a low weight. When the screen in FIG. 7 overlaps the screen in FIG. 8, each location should be represented by only one image from one of the screens. For example, the third image part M3 in FIG. 8 fills into the vacant area H in FIG. 7. On the other hand, if each location is associated with two images, for example, the third image part M3 in FIG. 8 overlaps with the second image part M2 in FIG. 7, it depends on assigned weights of the images, and the image with the higher weight (i.e., the second image part M2 in FIG. 7) is displayed.

### Beneficial Effects of the Embodiment

With the disclosed system and the disclosed method for generating an endoscopic image, through dividing, identifying, and combining image parts of the images of the surgical site, the image part in which the surgical site is blocked by the surgical instrument can be replaced with another image part in which the surgical site is not blocked by the surgical instrument, and the doctor can see the whole surgical site. Therefore, the system for generating the endoscopic image can be used without a microscope, so that space in the operating room can be reserved to benefit surgical operations.

Moreover, through the images captured from different viewing angles by the two image capturing elements 13, the processing device 3 replaces the first image part M1 in FIG. 6 with the third image part M3 from FIG. 8 to obtain the processed image M″. The processed image M″ is an unobstructed field image which is built by combining multiple surgical images from different angles. Therefore, through the system and method for generating the endoscopic image provided by the present disclosure, the image of the surgical instrument U is removed, and the image that is blocked by the surgical instrument U is revealed.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A system for generating an endoscopic image, comprising:
an endoscope assembly for being used with a surgical instrument, wherein the endoscope assembly includes a plurality of image capturing elements;
a robot device connecting with the endoscope assembly, wherein the robot device is configured to move the endoscope assembly to vicinity of a surgical site, such that the plurality of image capturing elements capture a plurality of current images from different viewing angles, respectively, and one of the current images includes a first image part and a second image part; wherein the first image part is an image of a part of the surgical instrument, the second image part is an image of a part of the surgical site that is not blocked by the part of the surgical instrument, at least one of the current images further includes a third image part, and the third image part and the first image part correspond to a same area or a same position of the surgical site; and
a processing device electrically connected to the endoscope assembly, wherein the processing device is configured to:
identify positions and contours of the surgical instrument in the plurality of current images;
divide the plurality of current images to separate the first image parts, the second image parts, and the third image parts of the plurality of current images; and
replace one of the first image parts with one of the third image parts, so as to obtain a processed image.

2. The system for generating the endoscopic image according to claim 1, wherein the process of replacing the first image part with the third image part to obtain the processed image further includes:
comparing the second image parts of the plurality of current images, and selecting one of the current images as a main image; and
overlapping the current images, wherein:
if the first image part of the main image overlaps with the third image part of another current image, the first image part is replaced with the third image part of the another current image; or
if the third image part of the main image overlaps with the third image part of another current image, the third image part of the main image is retained for display.

3. The system for generating the endoscopic image according to claim 1, wherein the endoscope assembly further includes an outer sleeve and an inner sleeve that are detachable from each other, the outer sleeve has a first opening and a second opening that communicate with each other, the inner sleeve is disposed at the first opening, the plurality of image capturing elements are disposed on the inner sleeve and distributed around the inner sleeve, and the plurality of image capturing elements faces the second opening of the outer sleeve.

4. The system for generating the endoscopic image according to claim 1, further comprising a plurality of navigation marker frames that are deformable, wherein the plurality of navigation marker frames are disposed on the robot device, the endoscope assembly, and the surgical site for establishing a spatial coordinate system; wherein the robot device is configured to guide the surgical instrument to move to vicinity of the surgical site based on the spatial coordinate system, and through the spatial coordinate system and the endoscope assembly, the processing device obtains position information of the surgical instrument in the surgical site and generates the processed image corresponding to the position information.

5. A method of generating an endoscopic image, comprising:
providing a robot device to connect an endoscope assembly and move the endoscope assembly to vicinity of a surgical site, wherein the endoscope assembly is suitable for being used with a surgical instrument;
providing a plurality of image capturing elements that are disposed on the endoscope assembly to respectively capture a plurality of current images of the surgical site from different viewing angles, and one of the current images includes a first image part and a second image part, wherein the first image part is an image of a part of the surgical instrument, the second image part is an image of a part of the surgical site that is not blocked by the part of the surgical instrument, at least one of the current images further includes a third image part, and the third image part and the first image part correspond to a same area or a same position of the surgical site; and
providing a processing device that is electrically connected to the endoscope assembly to:
identify positions and contours of the surgical instrument in the plurality of current images;
divide the plurality of current images to separate the first image parts, the second image parts, and the third image parts of the plurality of current images; and
replace one of the first image parts with one of the third image parts, so as to obtain a processed image.

6. The method of generating the endoscopic image according to claim 5, wherein the process of replacing the first image part with the third image part to obtain the processed image further includes:
comparing the second image parts of the plurality of current images, and selecting one of the current images as the main image; and
overlapping the current images, wherein:
if the first image part of the main image overlaps with the third image part of another current image, the first image part is replaced with the third image part of the another current image; or
if the second image part of the main image overlaps with the third image part of another current image, the second image part of the main image is retained for display.

7. The method of generating the endoscopic image according to claim 6, wherein the endoscope assembly further includes an outer sleeve and an inner sleeve that are detachable from each other, the outer sleeve has a first opening and a second opening that communicate with each other, the inner sleeve is disposed at the first opening and is located inside the outer sleeve, the plurality of image capturing elements are disposed on the inner sleeve and distributed around the inner sleeve, and the plurality of image capturing elements faces the second opening of the outer sleeve.

8. The method of generating the endoscopic image according to claim 7, wherein the process of moving the endoscope assembly to the surgical site further includes:
calibrating a plurality of lenses of the plurality of image capturing elements; and
assembling the inner sleeve onto the outer sleeve.

9. The method of generating the endoscopic image according to claim 8, wherein, before the process of assembling the inner sleeve onto the outer sleeve, the method further includes:
assembling a plurality of deformable navigation marker frames onto the robot device, the outer sleeve, and the surgical site to establish a spatial coordinate system;
utilizing the processing device to plan a surgical path based on the spatial coordinate system, so as to determine a position and an angle at which the outer sleeve moves to vicinity of the surgical site; and
utilizing the robot device to move the outer sleeve vicinity of the surgical site based on the surgical path.

10. The method of generating the endoscopic image according to claim 9, wherein, after the process of utilizing the robot device to move the outer sleeve to the surgical site based on the surgical path, the method further includes:
utilizing the robot device to obtain position information of the surgical instrument in the surgical site and the processed image corresponding to the position information through the spatial coordinate system and the endoscope assembly.
